(19) United States
(12) Patent Application Publication
(10) Pub. No.: US 2003/0113812 A1
(43) Pub. Date: Jun. 19, 2003

(54) Proliferation and differentiation of stem cells using extracellular matrix and other molecules

(76) Inventor: John J. Hemperly, Apex, NC (US)

Correspondence Address: BECTON, DICKINSON AND COMPANY, 1 BECTON DRIVE, FRANKLIN LAKES, NJ, 07417-1880, US

(21) Appl. No.: 10/259,815

(22) Filed: Sep. 30, 2002

Related U.S. Application Data
(60) Provisional application No. 60/326,440, filed on Oct. 2, 2001.

(51) Int. Cl. G01N 33/53; B05D 3/00; G01N 33/567; G01N 33/574; C12M 1/34; C12N 5/08

(52) U.S. Cl. 435/7.2; 435/7.23; 435/368; 435/287.1; 427/2.11

(57) ABSTRACT

Methods and compositions for testing agents for their effects on growth and differentiation of cells, primarily stem cells of various origin, are disclosed. Also disclosed are methods for inducing growth and differentiation of bone marrow stem cells primarily along the pathway to neuronal progenitor cells.

### BACKGROUND OF THE INVENTION

1. Field of the Invention

The present invention is directed to methods which can be used to test agents in cell culture for their effect on cell growth, differentiation and other activities. Additionally, this invention is directed to the use of specific agents to induce bone marrow stem cells to grow and differentiate in culture, primarily into neuronal progenitor cells (“NPC”).

The goal of tissue engineers is to meet these needs by creating living, three-dimensional tissues and organs using cells. In many cases, the approach is to coax cells into forming a tissue structure of the appropriate size and/or shape using a physical scaffold to organize cells on a macroscopic scale and provide molecular cues to stimulate appropriate cell growth, migration and differentiation. For example, in some applications, such as bone and blood vessel engineering, the donor material may be progenitor cells which can be stimulated to migrate, proliferate and differentiate, and then form appropriate tissue structures within a scaffold implanted into a site in the body.

2. Description of the Background Art

A stem cell is a cell that has the ability to divide (replicate itself) for indefinite periods and to give rise (differentiate), under the right conditions, to the many different cell types that make up an organism. That is, stem cells have the potential to develop into mature cells that have characteristic morphology and specialized functions, such as heart cells, skin cells, nerve cells, etc.

Embryonic stem (ES) cells are able to differentiate into every cell type of an organism. Unlike other stem cells, they can differentiate into cells that are derived from all three primary germ layers: the ectoderm, mesoderm or endoderm. Each cell type and tissue type in an adult organism originates from these three primary germ layers. The endoderm is the source of epithelial cells lining respiratory passages and the gastrointestinal tract and gives rise to the pharynx, esophagus, stomach and intestine and many glandular tissues and organs, including salivary glands, liver, pancreas and lungs. The mesoderm gives rise to smooth muscle cells, connective tissue, and various vessels coursing through tissues and organs. The mesoderm forms most of the cardiovascular system and is the source of bone marrow and blood cells, the skeleton, striated muscles and the reproductive and excretory organs. The ectoderm forms the epidermis, the sense organs and the entire nervous system, including brain, spinal cord, and all the peripheral nerves.

Adult stem cells which have been identified in the bone marrow, peripheral blood, brain, spinal cord, dental pulp, blood vessels, skeletal muscle, epithelia of the skin and digestive system, cornea, retina, liver and pancreas, have more limited potential than ES cells and are usually committed to differentiate into cells that contribute to the function of the tissue from which they originated. For example, adult stem cells from the brain (neural stem cells) give rise to neurons and glial cells; adult stem cells from the skin give rise to basal cells, squamous cells and melanocytes; and blood (or hematopoietic) stem cells give rise to red and white blood cells and platelets.

Recent studies have identified adult stem cells with the potential to differentiate into the specialized cells of unrelated tissues under certain conditions, including cells corresponding to a tissue derived from the same or from a different embryonic germ layer. See for example, Orlic et al. Nature 410:701-705 (2001), Gussoni et al. Nature 401:390-394 (1999), Bjornson et al., Science 283:534-537 (1999). For example, blood stem cells (mesodermal origin) can under certain circumstances generate skeletal muscle cells (also mesodermal origin) and neurons (ectodermal origin).

Based on the foregoing, such adult stem cells may, therefore, ultimately be used as a renewable source of cells that differentiate into a variety of progeny useful for treating a number of diseases and deficiencies. One particularly important use is the treatment of neurological diseases such as Parkinson's disease (“PD”). Unfortunately, neural stem cells are not a particularly abundant source because they reside deep in the brain, severely constraining accessibility for harvesting. Conversely, bone marrow (BM) stem cells are more abundant and accessible. The ease with which bone marrow stem cells are harvested by simple marrow aspiration, makes them excellent candidates for therapeutic use.

BM comprises a number of stem cell types. Best known among these are hematopoietic stem cells (HSCs) and marrow stromal cells (MSCs). In normal mammals, HSCs give rise to blood cells whereas MSCs give rise to cell types that populate other tissues and sites such as cartilage or bone, hematopoietic supportive stromal cells and fat. Recent studies have suggested that these BM stem cells can, under certain conditions, differentiate into additional cells types such as cardiac myocytes, liver cells, and skeletal muscle cells. Additionally, BM stem cells have been shown to have the potential for generating neurons (Sanchez-Ramos et al. Exp. Neurol. 164 247-256 (2000), Woodbury et al. J. Neurosci. Res. 62: 364-370 (2000), Mezey et al. Science 290: 1779-1782 (2000), Brazelton et al. Science 290: 775-1779 (2000). Chopp's group has investigated the use human MSCs (hMSCs) to treat rats subjected to strokes. Li Y et al., Neurology, 2002, 59:514-523, tested the effect of intravenously administered hMSCs on neurologic functional deficits after stroke. Treatment with hMSC resulted in significant recovery of function at 14 days compared with control rats with ischemia. Neurologic benefit resulting from this hMSC treatment appeared to derive from the increase of growth factors in the ischemic tissue, the reduction of apoptosis in the penumbral zone of the lesion, and the proliferation of endogenous cells in the subventricular zone. In a more recent publication from the same group, Chen X et al., J Neurosci Res, 2002, 69:687-691, investigated the temporal profile of various growth factors including brain-derived neurotrophic factor (BDNF), nerve growth factor (NGF), vascular endothelial growth factor (VEGF), basic fibroblast growth factor (bFGF), and hepatocyte growth factor (HGF), within cultures of human MSCs (hMSCs) conditioned with cerebral tissue extracts from traumatic brain injury (TBI). hMSCs in such cultures responded by producing more BDNF, NGF, VEGF, and HGF, supporting the notion that transplanted hMSCs provide therapeutic benefit in part via a responsive secretion of an array of growth factors that can foster neuroprotection and angiogenesis.

Laboratory grown cells derived from a several stem cell types, including BM-derived stem cells, may be a desirable source of transplantable material for grafting into brains of individuals suffering from neurological disorders (Web Address: nih. gov/news/stemcell/scireport.htm, (June 2001).

To induce stem cells to differentiate, it is desirable to identify the right combination of molecules and cell-culture conditions to (a) support survival and/or self-renewal of undifferentiated cells in culture and (b) stimulate them to become committed to a desired cell lineage such as neurons. Such cells may then be implanted into an appropriate site in vivo to complete their growth and differentiation program.

Although MSCs substantially purified from HSCs can differentiate into NPCs in vitro, HSCs only appear to undergo such a program when they are a component of BM stem cells and then, only in vivo. It would be desirable to discover whether HSCs develop neuronal characteristics in vitro and to determine the factors and conditions for such differentiation.

The process of HSC (or other stem cell) differentiation into particular progeny in vitro requires the action of many factors, including growth factors, extracellular matrix (“ECM”) molecules and components, environmental stressors and direct cell-to-cell interactions. The appropriate agents that will enhance or direct stem cell differentiation along a particular path, however, may be difficult to predict.

For example, when human “leukemia inhibitory factor” (hLIF) was added to cultures of human MSCs, these cells developed fibroblastic morphologies (Sanchez-Romos et al.). The same protein, however, had been shown to be essential for maintaining mouse ES cells in an undifferentiated state (Sanchez et al., 2000). This illustrates the difficulty in knowing in advance the effect of a particular molecule on a particular cell type.

Woodbury et al. initiated neuronal differentiation by culturing MSCs in medium containing β-mercaptoethanol. This molecule, however, did not result in “optimal” differentiation as the number of cells undergoing differentiation varied markedly within and between experiments. Pretreatment of these cells with the growth factor, bFGF gave more consistent results.

Currently available methods for assaying cell culture conditions as they influence cell growth and differentiation do not enable easy and rapid assessment of the relative effects of a variety of agents at various concentrations. Commonly, cells are grown in adjacent wells of a tissue culture plate in a medium comprising different components and then are tested, for example, for the presence of markers indicative of differentiation. Subtler effects of, for example, a cell signaling factor that induces a slight change in morphology, may not be readily observed or discerned when samples are in separate wells. Because a large number of agents need to be tested to optimize cell culture conditions, it would be beneficial to have a way to quickly eliminate suboptimal factors without resorting to extensive testing. Additionally, culture in separate wells or vessels prevents cells from migrating toward or away from an agent that might act on them. Such migration could enable cell-to-cell signaling that could lead to differentiation to a desirable phenotype. It would be useful to have a general method that permits quick and facile observation and discrimination of more subtle as well as more dramatic changes in stem cell growth and differentiation in response to a variety of signaling factors. (Such methods, described herein, can be found in greater detail in copending commonly assigned U.S. Patent Application Ser. No. ______, Heidaran et al., filed on even date herewith, and based on U.S. Provisional application No. 60/335,898, all of which are incorporated by reference in their entirety.)

Additionally desirable is a method that enables differentiation of HSCs into NPCs as it would enhance the usefulness of these stem cells in the treatment of neurological diseases.

### SUMMARY OF THE INVENTION

The present invention is directed in part to a method for inducing differentiation of stem cells of various types into NPCs based on the use of methods that evaluate and identify potential inducing agents that induce such differentiation.

More generally, this invention provides a method testing a plurality of potential inducing agents or different concentrations of a potential inducing agent on a single cell culture surface for effects on cell growth and/or differentiation. The method includes forming a support surface having potential inducing agents thereon in the form of at least one CAR region and at least one bioactive region, which terms are defined below, and depositing cells onto each of said regions and determining the effects on cells that are in contact with each of said regions and thereby exposed to the bioactive molecules that constitute the bioactive region.

Related to the foregoing, the present invention is directed to compositions and methods in which different bioactive regions are juxtaposed on a single surface or in a single culture vessel, such that cells can respond to differences or gradients between regions. For example, juxtaposed bioactive regions may comprise different bioactive agents, or different concentrations of the same bioactive agent such that, if a concentration gradient of a particular bioactive agent can serve as an inducing signal for a cellular activity, for example, growth or differentiation, the cell can respond accordingly to such a gradient. Conditions such as these attained by the present invention would not occur in or on conventional culture surfaces or vessels when employing conventional cell culture methods. Thus, the present approach permits detection of signals and interactions that may be important in vivo but that are lost in the conventional cell/tissue culture environment.

Examples of gradients impacting cellular differentiation, development and function are well known in the art, particularly in developmental biology. Tabata T, Nature Rev Genet, 2001, 2:620-630, described how organization of cells and tissues is controlled by the action of “form-giving” signalling molecules, termed morphogens, which pattern a developmental field in a concentration-dependent manner. The concentration gradient of the morphogen prefigures the pattern of development. During mammalian pituitary gland development (Scully KM et al., Science, 2002, 295:2231-2235), distinct cell types appear in response to opposing signaling gradients that emanate from distinct organizing centers. These signals induce expression of interacting transcriptional regulators in temporally and spatially overlapping patterns. Together they synergistically regulate precursor proliferation and induction of distinct cell types. To simplify the orchestration of development, organisms use the strategy of separating cell populations into distinct functional units wherein fields of cells are subdivided by the “interpretation” of morphogen gradients, and these subdivisions are then maintained and refined by local cell-cell interactions (Irvine K D et al., Ann Rev Cell Devel Biol, 2001, 17:189-214). Once cell populations become distinct, specialized cells are often induced along the borders between them. These boundary cells can then influence the patterning of surrounding cells, which can result in progressively finer subdivisions of a tissue. Christoffels V M et al., Hepatology, 1999, 29:1180-1192, disclosed how, in the liver, genes are expressed along a portocentral gradient. Based on their adaptive behavior, a gradient versus compartment type gradient has been recognized. These authors tested a model that used portocentral gradients of signal molecules as input and output that depended on two gene-specific variables, the affinity of the gene for its regulatory factors and the degree of cooperativity that determined the response in the signal-transduction pathways. Interaction between two or more different signal gradients may be necessary to ensure a stable expression pattern under different conditions. All of the foregoing types of interactions are difficult to mimic in vitro in conventional system, but are examples of the types of activities that the present invention encompasses by its recognition of the utility of distinct regions of biologically active molecules, such as ECM components, juxtaposed in a pre-determined manner in or on a single cell culture surface.

Thus, the invention also provides a method for inducing differentiation of stem cells into neuronal progenitor cells that comprises contacting stem cells with ECM molecules, permitting the stem cells to differentiate into neuronal progenitor-like cells; and detecting this differentiation. Also included is a method for inducing differentiation of osteogenic cells.

Preferred inducers of NPC differentiation are the following combinations of polypeptides with either ECM molecule or other non-ECM polypeptides: poly-L-ornithine (PLO)/laminin, PLO/fibronectin, PLO/collagen VI, PLO/vitronectin, poly-L-lysine (PLL)/collagen VI and PLL/vitronectin. Additionally, the PLO/PLL combination was effective. The poly-D-amino acid isomers of the foregoing may also be used.

In one embodiment, the method for inducing or promoting the differentiation of stem cells into neuronal progenitor cells comprises:

(a) contacting stem cells in a culture vessel having a surface with extracellular matrix molecules that are capable of inducing stem cell differentiation to neural progenitor cells; and

(b) culturing the stem cells for a time sufficient to permit them to differentiate into neuronal progenitor cells.

The method may further comprise a step of detecting the differentiation.

Also provided is a method for producing an isolated or enriched population neuronal progenitor cells from stem cells, comprising:

(a) contacting stem cells in a culture vessel having a surface with extracellular matrix molecules that are capable of inducing stem cell differentiation to neural progenitor cells;

(b) culturing the stem cells for a time sufficient to permit them to differentiate into neuronal progenitor cells;

(c) optionally, detecting the differentiation; and

(d) enriching or isolating the neuronal progenitor cells, using any known means.

In the foregoing method, the surface is preferably coated with a layer of a cell adhesion resisting (CAR) material or agent, which may be bonded directly to the surface or indirectly, via binding to an intermediate layer which is bonded to the surface.

Preferred CAR materials include(a) polyethylene glycol, (b) glyme, (c) a glyme derivative, (d) poly-HEMA, (e) polyisopropylacrylamide, (f) HA, (g) AA and (h) a combination of any of (a)-(g). Most preferred is HA.

The above intermediate layer may be selected from the group consisting of polyethyleneimine (PEI), poly-L-lysine (PLL), poly-D-lysine (PDL), poly(vinylamine) (PVA), and poly(allylamine) (PAA). PEI is preferred.

The foregoing method may be carried out with stem cells, including bone marrow stem cells (including marrow stromal cells), and pluripotent stem cells. It should be noted that these methods can also be carried out using any culturable cell, including long term cell lines that, preferably, are capable not only of growing in culture but which can differentiate in response to one or more inducing agents.

The steps for detecting differentiation of NPCs comprise observing or measuring nestin or another marker of NPCs in or on the surface of cells that have be stimulated to differentiated along an NPC pathway in the culture. Preferably, nestin is measured using a specific anti-nestin antibody.

This invention is also directed to an article useful for evaluating a candidate agent for its cell growth-inducing or cell differentiation-inducing activity, comprising:

(a) a support surface coated with a CAR material having bound thereto at least one bioactive agent, such that the resulting surface comprises at least one exposed CAR region and at least one exposed bioactive region, wherein the CAR material, the at least one bioactive agent or a combination of both is the candidate agent.

In the above article, the at least one CAR region preferably comprises a CAR material selected from the group consisting of (a) polyethylene glycol, (b) glyme, (c) a glyme derivative, (d) poly-HEMA, (e) polyisopropylacrylamide, (f) hyaluronic acid, (g) alginic acid and (h) a combination of any of (a)-(g). The CAR region may be a tissue culture-treated surface to which the CAR material is bonded.

The article preferably has at least one bioactive region comprising a bioactive agent which is an ECM molecule or a growth factor. Preferred ECM molecules are laminin, vitronectin, fibronectin, elastin, collagen I, collagen III, collagen IV, collagen VI, entactin, a proteoglycan, or Matrigel™.

In this article the CAR region may be bonded to the surface through an additional “intermediate” which is bonded directly to the surface, and is preferably a material that is noted above.

In the foreoing method and article, when the bioactive region comprises a growth factor, the growth facotor is preferably a bone morphogenetic proteins, epidermal growth factor, erythropoietin, heparin binding factor, hepatocyte growth factor, insulin, insulin-like growth factor I or II, an interleukin, a muscle morphogenic protein, nerve growth factor, platelet-derived growth factor, or transforming growth factor α or β.

In the article, the bioactive region may be in the form of a plurality of spots, with each spot comprising at least one bioactive agent deposited thereon. Alternatively, the at least one bioactive region may be in the form of a plurality of spots, with each spot comprising a different concentration of one bioactive agent. The plurality of spots maybe arrayed in a grid pattern on the surface.

### BRIEF DESCRIPTION OF THE DRAWINGS

FIGS. 1A-1B show growth of rat BM cells on polystyrene surfaces coated with a cell adhesion resistant (CAR) material, HA (in oxidized form), and on spots of Matrigel™ (abbreviated MG in the figures) which was bound covalently to the oxidized HA surface. The MG used here was a 1:16 dilution. FIG. 1A is a phase contrast photomicrograph of the cells growing on MG or, less densely, on oxidized HA. The curvature of the MG spot can be discerned; cells are denser at the edge of the spot. FIG. 1B shows the same cells with a murine monoclonal antibody (mAb) specific for rat nestin. Nestin-positive cells are stained dark in this photograph. The presence of nestin is visualized by immunofluorescence using a rhodamine-labeled second antibody (anti-mouse immunoglobulin). Similar results were obtained using a 1:4 dilution of MG.

FIGS. 2A-2C show differentiation of MC3T3 cells, an osteogenic stem cell-like cell line, on an oxidized HA surface but not on an MG surface. Murine MC3T3 cells were cultured for 11 days on oxidized HA onto which had been spotted various dilutions of MG. Cells were fixed with formaldehyde and processed to visualize alkaline phosphatase, an enzyme produced by cells that have differentiated to a more bone-like phenotype. Although there were patches of alkaline phosphatase-positive cells at multiple locations in the oxidized HA region, no alkaline phosphatase-positive cells were observed on the ECM spots, despite more rapid cell growth on these spots. The pre-spotted ECM proteins, in the form of MG, inhibit bone differentiation of MC3T3 cells.

### DESCRIPTION OF THE PREFERRED EMBODIMENTS

Conventional molecular biological or cell biological techniques are disclosed, for example, in the following references: Sambrook et al., Molecular Cloning: A Laboratory Manual (1989); Current Protocols in Molecular Biology Volumes I-III (F. Ausubel, ed. (1994)); Cell Biology: A Laboratory Handbook “Volumes I-III (J. E. Celis, ed. (1994); Oligonucleotide Synthesis (M. J. Gait, ed., 1984); Nucleic Acid Hybridization (B. D. Hames et al., eds., (1985); Transcription and Translation (B. D. Hames et al., eds (1984)); Animal Cell Culture (R. I. Freshney, ed., (1986)); Immobilized Cells and Enzymes” (IRL Press, (1986); and B. Perbal, A Practical Guide to Molecular Cloning (1984).

The term “differentiation” is well-known in the art, and as used herein, is intended to be broad and to include the potential of any and all types of stem or progenitor cells to produce more specialized or mature or committed progeny cells.

The term “growth,” as used herein, means any increase in cell number, cell size or in the quantity or concentration of a cellular component such as an organelle and/or an elongation of a cellular “process.” Cellular processes are extensions of the cytoplasm and may include specialized stuctures; examples include axons, dendrites, pseudopods, cilia, sensory endings, and flagella.

An “inducing agent” or “induction agent” is a substance which acts to promote growth or differentiation of cells.

A low affinity material or agent is also termed “cell-adhesion resisting” or “cell-adhesion resistive” (“CAR”) material or agent herein because in its presence, cell adherence or attachment to a surface is inhibited or prevented. Based on the properties of these materials, certain macromolecules are also less likely to bind to a CAR surface. According to the present invention, an inducing agent may be provided in the form of a surface or region of a cell culture device or vessel. Cell growth and/or differentiation may be promoted or inhibited by the properties which have been conferred on the surface which render the surface CAR and/or bioactive. A low affinity or CAR material is one that does not generally enable, and preferably inhibits, cell attachment or adherence. Thus, a CAR material attached to or coated on a surface creates and defines a region or zone of low affinity for cells (=adhesion resistance). Suitable CAR materials include but are not limited to polyethylene glycol, glyme and derivatives thereof, poly-HEMA, poly-isopropylacrylamide and, preferably any of a number of polysaccharides including hyaluronic acid (HA) and alginic acid (AA). In a more preferred embodiment, HA is used as a CAR material. In general, highly hydrophilic substances containing a high concentration of hydroxyl groups may be used as CAR materials, either alone or in combination. A CAR material or the CAR surface may act as an inducing agent either (a) alone, (b) in combination with other CAR materials or (c) in combination with bioactive agents that are not CAR materials. The effects of such bioactive agents may be tested using methods and assays, described herein and well-known in the art, for their effects on a selected cellular function such as cell growth or differentiation.

A “low affinity region” or CAR region is an area on a support surface onto which a CAR material has been placed, added, spotted, etc. A first region is “juxtaposed” to a second region if the two regions are adjacent to one another on a surface, or, are sufficiently close to one another that cells in or on the first region can respond to signals the second, juxtaposed region or to a concentration gradient between two juxtaposed regions. Two juxtaposed regions may be in direct contact so that no other surface intervenes, or may be spaced at varying distances from one another. For example, two bioactive regions that are coated onto a CAR surface may abut one another or, alternatively, may be spaced so that CAR surface that is not coated with a bioactive material separates the two.

Any geometric relationship between CAR regions and bioactive regions is included in the scope of this invention. Thus, one preferred surfaces is coated with a uniform CAR layer which has placed upon it, preferably bonded covalently or bound noncovalently, bioactive regions that comprise single agents or mixtures of agents. If a single agent is used, neighboring regions may have different concentrations of that agent. The bioactive regions may abut one another with no spacing or may have unmodified areas of CAR surface between them. In another embodiment, discrete CAR regions are distributed on the surface; some of these are modified with a bioactive agent so that the surfaces includes CAR-only regions and bioactive regions (on a CAR surface). Such a surface may optionally include bioactive-only regions which are bound to the surface in the absence of an underlying CAR material. Such regions may be prepared on any suitable surface for use in cell culture or in cellular assays, and includes sheets, slides, dishes (e.g., petri dishes), culture flasks, multiwell cluster dishes of any number and geometric layout of wells. Preferred are multiwell plates having 96 wells, 192 wells, 384 wells, etc.

The particular CAR material used or the particular concentration of CAR material selected, may inhibit cell adhesion to a varying degree. For example, polystyrene coated with a 0.5% (w/v) solution of AA was found to prevent cell adherence after 3 days. With a 0.1% (w/v) solution, very little cell adherence was seen at 4 hours but increased substantially after 3 days of incubation (Morra, M. and C. Cassinella, J: Biomater. Sci.-Polymer Ed. 10:1107-1124 (1999), incorporated by reference in its entirety).

In one embodiment, materials having basic reactive groups such as amines or imines can be used as an intermediate layer or sublayer attached directly to the support surface beneath the CAR material. As used herein an “intermediate layer” or “sublayer” is a layer of material deposited onto a support surface and with which a CAR material reacts preferably forming covalent bonds. Examples of commercially available materials include PEI, PLL, PDL, PVA and PAA. PEI may be reacted ionically with a support surface to provide amino groups on the surface which are then coupled to a CAR material using carbodiimide coupling.

In another aspect of the invention, a CAR material is deposited onto a support surface which has been “tissue culture treated” without a need for an intermediate layer. As used herein, a “tissue culture treated” support surface is one that has been treated with a plasma discharge in a vacuum or with a corona discharge. For plasma discharge in a vacuum, molded parts of a support surface are placed in a vacuum chamber and a mixture of gases including oxygen is pumped in. Under defined conditions of a partial vacuum, an electrical discharge creates a reactive plasma which reacts with the support surface. This process creates negatively charged functional groups on the surface including hydroxyl, carbonyl and carboxyl groups. Mixtures of other gases can also be added to create a more complex tissue culture treated surface. For example, the surfaces of Primaria™ products (BD Biosciences, Bedford, Mass.) and CELL+products (Sarstedt, Newton, N.C.) contain both positively and negatively charged functional groups that can promote attachment of CAR substances.

In another embodiment, a sublayer or intermediate layer such as PEI may be deposited onto a tissue culture-treated support surface.

Alternatively, a CAR material may be placed or spotted onto only a portion of support surface. Such a geometry permits determination of whether the support surface itself or the sublayer acts as an inducing agent.

A “bioactive agent” as used herein is a substance, typically a molecule, which affects physiological cellular processes and which may permit or enhance cell adherence. A “bioactive region” is defined as an area, zone or region on a support surface that comprises an added bioactive agent. Bioactive agents generally include, but are not limited to, peptides, polypeptides (natural or synthetic), proteins, including antibodies and ECM molecules. Any lysate or extract of cells or a tissue can serve as a bioactive agent (thought such a preparation is in effect a collection of bioactive agents). Certain polyamino acids, such as poly-L-ornithine (PLO) and PLL are effective inducers of NPC differentiation when used in combination with one another or in combination with selected ECM molecules. Preferred combinations are: PLO/laminin, PLO/fibronectin, PLO/collagen VI, PLO/vitronectin, PLL/collagen VI and PLL/vitronectin.

ECM molecules (see, for example) Kleinman et al., J. Biomater. Sci Polymer Ed 5: 1-11, (1993), herein incorporated by reference) are well known to those skilled in the art. Non-limiting examples of ECM molecules are fibronectin, vitronectin, collagens, laminin, elastin, various proteoglycans, glycosaminoglycans and the like. Many ECM molecules are commercially available. For example, a very commonly used ECM material, Matrigel™ is made from the EHS mouse sarcoma tumor and is available from BD Biosciences, Bedford, Mass.

As noted, a bioactive agent may act as an inducing agent with certain cell types and at particular concentrations. However, a bioactive agents may also be inhibitors of cell growth, cell differentiation or other cellular functions.

In one embodiment of this invention, a bioactive region of a surface is compared with a CAR region of the same surface to determine if the bioactive agent inhibits growth and/or differentiation. In one embodiment, a bioactive agent acts as an inducing agent whereas a CAR material has no effect on cell differentiation. In another embodiment, an inducing agent or signal comes in the form of a gradient of one or more different bioactive regions that are juxtaposed on a single surface, such that cells can respond to the differences in bioactive agents or differences in concentration of a bioactive agent. Such induction is typically stimulatory to a cellular activity. However, in another embodiment a particular bioactive agent or a gradient created by juxtaposition of different bioactive regions acts to inhibit or prevent the induction of cell growth or differentiation (or other cellular activity).

Fragments or domains of larger molecules, typically macromolecules which are themselves bioactive agents, may also be bioactive agents as intended herein. Preferred fragments are extracellular domains of ligand-binding polypeptides, for example, a ligand-binding domains or fragment or region of an ECM molecule. These may be “adhesion domains” (defined below). A ligand that binds to cellular receptors or to such ligand-binding domains derived from receptors, may act as an inducing agent by promoting cell adherence as a result of binding to cell surface transmembrane protein. Transmembrane proteins transmit information and may carry molecules from outside the cell to the inside. Many of the transmembrane proteins are receptors, characterized by an extracellular ligand-binding domain and an intracellular signaling/regulatory domain. When a receptor binds a ligand, a change in receptor conformation or affinity for other molecules initiates an intracellular cascade of enzyme-mediated reactions resulting in amplification of the signal initiated by the extracellular binding event. This process is termed “signal transduction”. The surface of a typical mammalian cells includes dozens of different types of receptors, each with the capacity to trigger unique or common signal transduction pathways. Cellular functions including survival, proliferation, differentiation and apoptosis are governed by the integrated signals from numerous ligand molecules interacting with numerous cognate receptor molecules in a highly dynamic system.

Techniques used to understand the structural/functional properties of many ECM molecules have mapped receptor-binding functions to small “adhesion domains” of larger receptor proteins. As used herein an “adhesion domain” is a stretch of about 3 to about 20 amino acids of which the sequence is preferably conserved among different proteins (Griffith, L., Acta mater 48:263-277 (2000)). One prototypical adhesion domain is the tripeptide, arginine-glycine-aspartate (RGD), first identified as a minimal sequence required for cell adhesion to the ECM molecule fibronectin. RGD has since been found to be involved in cell adhesion to a wider array of ECM molecules. Other short adhesion-mediating peptide domains within ECM molecules have been identified and characterized (Griffith, supra). These adhesion domain peptides interact with a class of cell surface adhesion receptors called integrins. A functional integrin receptor comprises two subunits, an α chain and a β chain, drawn from a family of 16α chain and 8β members, which permits great diversity in the specificity of receptor-ligand interactions. Integrins mediate many aspects of cell behavior besides adhesionper se. Manipulation of integrin ligation by placing peptide adhesion domains on suitable surfaces can affect cell growth and differentiation in culture.

Other bioactive agents included herein are growth factors which may act synergistically with ECMs or other bioactive agents to affect adhesion, growth/proliferation, differentiation or other cellular behavior. Growth factors are typically characterized as relatively soluble (diffusible) peptides or polypeptides. Preferred examples include bone morphogenetic proteins (BMP), epidermal growth factor (EGF), erythropoietin (EPO), heparin binding factor (HBF), hepatocyte growth factor (HGF), insulin, insulin-like growth factor I or II (IGF-I, II), an interleukin, a muscle morphogenic proteins, nerve growth factor (NGF), platelet-derived growth factor (PDGF), transforming growth factor α or β (TGFα, TGFβ), and other factors known to those of skill in the art. See, for example, Sporn, MB et al., eds., Peptide Growth Factors and Their Receptors, Springer-Verlag, New York, (1990), which is herein incorporated by reference.

Growth factors can be isolated from tissue using conventional biochemical methods or produced by recombinant means in bacteria, yeast or mammalian cells (or other eukaryotic cells). For example, EGF can be isolated from the submaxillary glands of mice and TGF-β has been produced recombinantly (Genentech, S. San Francisco, Calif.). Many growth factors are also available commercially from vendors, such as Sigma Chemical Co. (St. Louis, Mo.), Collaborative Reasearch (Los Altos, Calif.), Genzyme (Cambridge, Mass.), Boehringer (Germany), R&D Systems (Minneapolis, Minn.), and GIBCO (Grand Island, N.Y.), in both natural and recombinant forms.

Other useful bioactive agents molecules include cytokines, such as the many interlukins, that may not be growth factors per se, and peptide hormones. These are well-known in the art and most are commercially available.

In one embodiment, a bioactive agent or a combination of such agents are deposited onto a CAR surface and are preferably allowed to adhere thereto. In another aspect of the invention bioactive agents provided to cells in soluble form, e.g., as a supplement to the culture medium.

In one embodiment, for example, as a control when evaluating bioactive agents, cells are added to a standard, commercially available serum-containing growth medium without added growth factors. Alternatively, a serum free medium, supplemented as described herein is used.

Suitable support surfaces for use herein include, but are not limited to ceramic, metal or polymer surfaces. Most preferred are polymer support surfaces. Suitable support surfaces are in the form of vessels described above (plastic dishes, flasks, microtiter plates) as well as plastic tubes, sutures, membranes, films, bioreactors and microparticles. Polymer surfaces may comprise poly(hydroxyethylmethacrylate), poly(ethylene terephthalate), poly(tetrafluroethylene), poly(styrene), poly(vinyl chloride), poly(hexafluoropropylene), poly(trifluoroethylene), poly(vinylidine fluoride), poly(dimethyl siloxane) and other silicone rubbers. Glass support surfaces that include glycerol propylsilane bonded glass are also contemplated.

In one embodiment, once a CAR region is formed on the support surface, a bioactive agent is immobilized thereto using mild bioconjugation techniques known in the art (K. Mosback, Immobilized Enzymes and Cells, Part B, Academic Press, Orlando, Fla., 1987; G. T. Hermanson et al., Immobilized Affinity Ligand Techniques, Academic Press, San Diego, Calif., 1992; S. F. Karel et al., “The Immobilization of Whole Cells. Engineering Principles.” Chemical Eng. Sci. 40:1321 (1985).

To achieve this, a bioactive agent is preferably coupled covalently to HA. The HA is partially oxidized with a mild oxidant to convert some of the cis-diols to di-aldehyde moieties. These functional aldehyde groups can then form Schiff bases with the amino groups of bioactive agent. Examples of mild oxidants include potassium permanganate or, preferably, sodium periodate.

In a preferred embodiment, a bioactive agent is coupled to one or more CAR regions in the form of a circular spot, a rectangular spot, an ovoid spot or a spot of any other arbitrary shape. Preferably, a bioactive agent is deposited onto a CAR material in a “grid pattern”, i.e., arranged as relatively uniformly spaced, horizontal and perpendicular spots.

The bioactive agent may be covalently bonded to a surface comprising a CAR material to create multiple bioactive regions each having a different concentration of the same agent. In another embodiment, each bioactive region comprises a different bioactive agent or a combinations thereof. Any combination of grids or other patterns wherein the same or multiple different bioactive agents are spotted is intended.

A cell or cells may be deposited onto a surface displaying CAR materials and/or bioactive agents. Although any cell type may be used in the present method, including prokaryotic and eukaryotic cells, most preferred are mammalian cells, particularly from humans, rats, mice or bovine species. In one preferred embodiment, stem cells are used.

“Stem cells” are defined here as cells that have the ability to divide continuously in culture while also giving rise to specialized, differentiated cells. They are undifferentiated or relatively undifferentiated, lacking the morphology or markers characteristic of mature or differentiated cells. Stem cells are generally characterized by their developmental or differentiative potential. Thus truly “totipotent stem cells” have the capacity to become, the embryo, extraembryonic membranes and tissues, and all postembryonic tissues and organs.

“Embryonic stem cells” (also referred to as ES cells or ESCs) are a type of uncommitted, totipotent stem cell isolated from embryonic tissue. When injected into embryos, ESCs can give rise to all somatic cell lineages as well as functional gametes. In the undifferentiated state, ESCs are alkaline phosphatase-positive, express immunological markers characteristic of embryonic stem and embryonic germ cells, express telomerase and retain the capacity for extended self renewal. Upon differentiation, ESCs become a wide variety of cell types of ectodermal, mesodermal and endodermal origin. ESCs have been isolated from the blastocyst inner cell mass or from gonadal ridges of mouse, rabbit, rat, pig, sheep, primate, including human, embryos. (See for example, Thomson et al. Proc. Natl. Acad. Sci. USA 92 7844-7848 (1995), Thomson et al., Science 282:1145-1147 (1998); Shamblott et al., Proc. Natl. A cad. Sci. USA 95 13726-13731(1998), all of which are herein incorporated by reference).

While a majority of the cells in an organism are the result of cellular progression through the sequence of development and differentiation, a few cells appear to leave this pathway to become reserve stem cells that contribute to ongoing maintenance of a stem cell pool that can aid in “repair” of an organism as needed. Such cells are referred to here as “adult stem cells” (ASCs). ASCs include cells known as “progenitor stem cells” as well as “pluripotent stem cells.” “Progenitor stem cells” are those cells that are committed to a particular lineage and, as such, give rise to progeny of a single lineage within their respective germ layers, e.g., thyroid (endodermal origin); muscle, bone (mesodermal origin), neurons, melanocytes, epidermal cells (ectodermal origin). ASCs can remain quiescent and nonreplicating. In contrast, lineage-committed progenitor stem cells are capable of self-renewal but may have a limited life-span before programmed senescence manifests itself.

Progenitor stem cells can be further classified as multipotent, oligopotent or unipotent. As used herein “multipotent progenitor cells” form multiple cell types within a lineage. “Unipotent progenitor cells” form cells of a single type. “Oligopotent stem cells” form cells of more than one type, but not all possible types, within a lineage.

To illustrate, the mature central nervous system (CNS) comprises three primary cell types: neurons, astroglia and oligodendroglia. Unipotent neural progenitor cells (NPCs) give rise solely (and invariably) to a single type of neuron or to astroglia cells or to oligodendroglial cells. Oligopotent NPCs can give rise to (a) neurons of a number of different neuronal phenotypes (e.g., sensory or motor neurons) but not to astroglia or (b) one type of neuron and one type of glial cell, or (c) astroglia and oligodendroglia but not neurons. In contrast, a “multipotent” NPC generates progeny cells of all three CNS lineages.

Non-limiting examples of progenitor stem cells include unipotent myosatellite myoblasts of muscle; unipotent adipoblast cells of adipose tissue, unipotent chondrogenic cells and osteogenic cells of the perichondrium and periosteum, respectively; oligopotent adipofibroblasts of adipose tissue; and oligopotent adipofibroblasts of adipose tissue.

NPCs also termed “Neuronal progenitor-like cells” (NPLCs) are cells are characterized by the expression of nestin, an intracellular intermediate filament protein. MAbs specific for rat nestin have been produced, e.g., RAT 401, (Hockfield, S. et at. J. Neurosci. 5(12):3310-3328 (1985). A polyclonal rabbit anti-nestin antiserum has been reported to recognize mouse nestin (Reynolds, D. A. et al. Science 255:1701-1710, (1992).

“Pluripotent stem cells” are stem cells that are capable of giving rise to tissues derived from more than one embryonic germ layer. Pluripotent stem cells are not committed to any particular tissue lineage (“lineage-uncommitted”) and can give rise to cells of endodermal origin and/or mesodermal origin and/or ectodermal origin. Pluripotent cells can remain quiescent, and they can be stimulated to proliferate and are capable of extensive self-renewal while remaining lineage-uncommitted. Pluripotent stem cells can generate various lineage-committed progenitor cells from a single clone at any time during their life span. Lineage-commitment occurs under the influence or one or more inducing agents. Once induced to commit to a particular tissue lineage, pluripotent cells assume the characteristics of lineage-specific progenitor cells.

In one embodiment of the present methods, CAR materials and bioactive agents are tested for their ability to act as stem cell inducing agents.

Non-limiting examples of pluripotent stem cells are the stem cells from the CNS, hematopoietic stem cells (HSCs) from bone marrow, peripheral blood and umbilical cord blood; and marrow stromal cells from BM. As used herein, the term “bone marrow stem cells” includes all stem cells derived from the BM.

The HSCs are those cells which are able to differentiate into all blood cell types. HSCs have also been shown to differentiate in vivo into non-blood cell types including liver cells and neuronal cells. HSCs can be identified, isolated and/or purified using single surface markers or combinations thereof. Undifferentiated HSCs express markers including c-kit, CD34 and MHC class I (e.g., in mice, H-2K) and lack known lineage markers. These cells are referred to as “Lin-negative” or “Lin⁻”. Two kinds of HSCs are known. Long-term HSCs proliferate (undergo self renewal) for the lifetime of an animal. Short-term HSCs proliferate for a limited duration. Long-term HSCs have high levels of telomerase activity. Telomerase is an enzyme that helps maintain the length of chromosome (telomeres), by adding nucleotides to the ends. Presence of telomerase activity is characteristic of undifferentiated, dividing cells and of cancer cells. Differentiated, human somatic cells have no detectable telomerase activity. Short-term HSCs differentiate into lymphoid and myeloid precursors for the two major lineages of white blood cells. Lymphoid precursors differentiate, inter alia, to T cells, B cells and natural killer cells. Myeloid precursors differentiate to monocytes and macrophages, neutrophils, eosinophils, basophils, megakaryocytes and erythrocytes.

The present invention includes use of HSCs, preferably long-term HSCs. Preferably, HSCs derived from BM are used. The BM cells may be use in unfractionated form. In other embodiments, HSCs from BM are partially purified, e.g., at least about 80% pure, more preferably at least about 85% pure, and even more preferably, at least about 90% pure.

“Marrow stromal cells” (MSCs) refer to a subclass of non-hematopoietic stem cells from BM which, in vivo, give rise to osteocytes, chondrocytes, and adipocytes. MSCs can be separated from HSCs by their greater ability to adhere to plastic surfaces.

The present invention includes use of any stem cell or stem cell population, including clonal populations of stem cells.

The present invention illustrated with the example of rat BM stem cells but is intended to encompass all mammalian BM stem cells. Mammalian BM stem cells and their progeny can be isolated from the relevant tissues of humans, non-human primates as well as from equine, canine, feline, bovine, porcine, and lagomorph species.

Methods of isolating stem cells are well known in the art and can be found in, for example, U.S. Pat. No. 5,827,735; Young et al, In Vitro Cell Devel Biol 29A:723-736 (1993); Rogers et al., Amer Surgeon 61:1-6 (1995); Pate et al, Surgical Forum XLIV: 587-589, 1993.

The present invention also provides methods for detecting the presence of growth inducing agents or particular differentiation inducing agents, by their ability to elicit stem cell growth or lineage commitment. The present method may be used to further characterize a known inducing agent or to identify a new inducing agent.

In one embodiment of the method of the present invention, a plurality of bioactive agents are spotted onto a surface which had been prepared with a CAR material coupled thereto. Cells are placed onto this surface, and, after an appropriate interval,, are observed visually to determine if cell growth has occurred at bioactive regions or at CAR regions, thereby quickly ascertaining the effectiveness of the bioactive agent being tested as an inducing agent.

An important advantage of the present approach of creating multiple different regions in the same culture vessel (e.g., a single plate or a single well or a multiwell plate) is that cells are permitted to migrate to a location in the culture vessel which they prefer. For example, cells of a particular type could migrate to the spot or region that displayed the appropriate (1) bioactive agent or (2) CAR material, or (3) combination of bioactive and CAR materials, or (4) a region having a particular concentration of the bioactive agent or CAR material.

Cell proliferation can be assessed by various techniques known in the art, for example, by staining followed by microscopic observation, or by turbidimetric methods, spectrophotometric methods (including colorimetry and measurement of light absorbance at a particular wavelength), counting with an automated cell counter and/or automated plate counter, measurement of total cellular DNA and/or protein, impedance of an electrical field (e.g., Coulter counter), bioluminescence, production of carbon dioxide, oxygen consumption, adenosine triphosphate (ATP) production or the like.

Differentiation may be assessed by expression analysis (e.g., mRNA expression), immunological analysis and histochemical analysis, or combinations thereof. For example, to determine whether a particular bioactive agent, CAR material or combination thereof induces cell differentiation, one may analyze expression of nucleic acids corresponding to certain genes. Methods for such analysis are well known in the art and include amplification methods (e.g., polymerase chain reaction, strand displacement amplification, etc.) and hybridization of probe sequences such as in Northern analysis (Sambrook et al. supra). Northern blots examine the expression of known (or unknown) genes. Alternatively, it is possible to generate cDNA libraries or to observe differential display of genes that are expressed, for example, in stem cells, cells derived from stem cells, before and after exposure to known (or unknown) CAR material and/or bioactive agents as disclosed herein. For example, Northern blots were used to assess the presence of MRNA transcripts of myogenin and MyoDl genes as a measure of the induction of myogenesis in a pluripotent stem cell clone (see for example, WO 01/2167, incorporated by reference).

Additionally, assessment of the binding of an antibody to an antigen, the expression of which is characteristic of a cellular phenotype or differentiation stage, is used to assess differentiation. As used herein, an “antibody” is any immunoglobulin (Ig) molecule or an antigen-binding (or epitope-binding) fragment thereof. The term encompasses polyclonal, monoclonal, and chimeric antibodies (the latter of which are described in U.S. Pat. Nos. 4,816,397 and 4,816,567) as well as single chain antibody molecules (also known as scFv molecules) (Skerra, A. et al (1988) Science, 240: 1038-1041; Pluckthun, A. et al. (1989) Methods Enzymol. 178: 497-515; Winter, G. et al. (1991) Nature, 349: 293-299); Bird et al., (1988) Science 242:423; Huston et al. (1988) Proc. Natl. Acad. Sci. USA 85:5879; U.S. Pat. No. 4,704,692 (Ladner), U.S. Pat. Nos. 4,853,871, 4,946,778, 5,260,203, 5,455,030).

The present method comprises examining a cell sample using an immunoassay that employs a detectably labeled antibody sufficient to recognize and bind to a stem cell, differentiated progeny cells of stem cells, or tissues that comprise such stem cells or progeny.

Methods for producing polyclonal antibodies are well-known in the art. See, for example, U.S. Pat. No. 4,493,795 (Nestor et al.). A monoclonal antibody (mAb) or an Fab chain derived therefrom can be prepared using conventional methods and hybridoma technology. See, for example, Hartlow, E. et al., Antibodies—A Laboratory Manual, Cold Spring Harbor Laboratory Press, Cold Spring Harbor, N.Y. (1988), which is incorporated herein by reference.

In one embodiment, the presence of a differentiated cell expressing an epitope of interest will be detected by a detectably labeled primary antibody against that epitope or, preferably by an unlabeled primary antibody and a detectably labeled secondary antibody specific for the Ig isotype of the primary antibody. The presence of the detectably labeled antibody bound to the cell(s) is measured using any appropriate method that is specific for the particular type of label. This presence of antibody bound to the cell is indicative of differentiation. Use of a method that permits measurement of a bound antibody is also termed herein “visualization of the antibody”.

Antibody labels most commonly employed are radionuclides, enzymes, fluorescers which fluoresce when exposed to ultraviolet light, luminescers, and the like. Numerous suitable fluorescent agents useful as labels are known, including fluorescein, rhodamine, auramine, Texas Red, AMCA blue and Lucifer Yellow. Methods of conjugating these labels to proteins are well-known. Preferred radionuclides are selected from the group consisting of ³H, ¹⁴C, ³²P, 35S ³⁶Cl, ⁵¹Cr, ⁵⁷CO, ⁵⁹Fe, ¹²⁵I and ¹³¹I.

Enzyme labels are likewise useful and can be detected by any of the presently utilized colorimetric, spectrophotometric or fluorimetric techniques. The enzyme is conjugated to an antibody or a particle by reaction with bridging molecules such as carbodiimides, diisocyanates, glutaraldehyde and the like. Many enzymes are known as detectable labels for immunoassay, preferred enzymes being peroxidase, β-glucuronidase, urease and alkaline phosphatase.

Cells in culture can be treated with an antibody to a differentiation marker to determine if cell differentiation has occurred. Antibodies are known which identify cells as neurons, bone cells and the like. Non limiting examples of antibodies useful to detect markers indicative of a particular cells type include the following: mesodermal markers indicative of muscle (e.g.,. myogenin [F5D, Developmental Studies Hybridoma Bank (DSHB), sarcomeric myosin[MF-20 (DSHB)], fast skeletal muscle[MY-32, sigma] myosin heavy chain[A4.74], (DSHB), smooth muscle[smooth muscle alpha-actin, 1A4 (Sigma)], cartilage (collagens type-II [CIICI, DSHB], bone (bone sialoprotein [WVIDI, DSHB], endothelial cells (endothelial cell surface marker [H-Endo, Accurate]); endodermal markers (α-fetoprotein [HAFP, Chemicon] epithelial cell [HA4cI9, DSHB]) and ectodermal markers (e.g., neural precursor cells [FORSE-I, DSHB], nestin [RAT-401, DSHB] neurons [8A2,DSHB]), neurofilaments [RT97, DSHB].

Histochemistry can be used to assess cell morphology and differentiation. Such characteristics include round central areas and spidery cell processes or long polygonal cells with intracellular fibers as an indication of a neuronal phenotype; the presence of small rounded multinucleated or binucleated cells with a central nucleus and perinuclear vesicles indicate liver cells; the presence of multinucleated linear and branched structures indicate skeletal muscle; alkaline phosphatase activity may indicate bone differentiation. Calcium precipitation using silver nitrate may also be used to identify bone differentiation.

The method of this invention is useful for detecting subtle differences between two inducing agents when the agents are presented on a single support surface creating a more uniform environment with respect to other cell culture parameters and conditions. This enables comparison of growth and/or differentiation of cells under the influence of a plurality of inducing agents or combinations thereof, while maintaining other variables constant. This permits determination of which agent or combination of agents result in a desired outcome, e.g., a certain amount of growth or differentiation along a certain pathway or to a particular stage.

In a preferred embodiment, BM stem cells are contacted with ECM molecules resulting in the differentiation of the stem cells into NPCs.

In another embodiment, HA is coupled to the surface of a culture dish and ECM molecules are deposited onto the HA layer. Stem cells, preferably BM stem cells, are added to this material, resulting in differentiation into NPCs. The latter can be visualized using an antibody to nestin (a marker for this cell type).

In another embodiment of the foregoing methods, HSCs derived from BM are used to produce a cell population of at least about 90% purity prior to contacting with the modified surfaces and subsequent differentiation.

In yet another embodiment, the differentiated NPCs are enriched or isolated using conventional methods, including immunologically based methods that employ antibodies specific for NPC markers, e.g., nestin.

Having now generally described the invention, the same will be more readily understood through reference to the following examples which are provided by way of illustration, and are not intended to be limiting of the present invention, unless specified.

### EXAMPLE 1

### Bone Marrow-Derived Stem Cells Differentiate into Neuronal Progenitor Cells

This example demonstrates differentiation of a population of bone marrow stem cells (which is contaminated with fewer than about 10% MSCs) into NPCs when the stem cells are cultured on a surface bearing ECM molecules absorbed to immobilized oxidized HA. This example also shows increased proliferation of such HSCs under these conditions (and when compared with the same cells cultured on HA surfaces alone that are not supplemented with ECM molecules).

BM was harvested from the femurs of Brown Norway rats and suspended in phosphate-buffered saline (PBS). Cells were passed through a screen to remove or break up aggregates and were collected by centrifugation. Red blood cells were lysed by hypotonic ammonium chloride and nucleated cells collected by centrifugation. These cells were resuspended in 5 ml of 10% FBS in Dulbecco's-modified Eagle's Medium (DMEM) supplemented with penicillin and streptomycin. Cells were enumerated by hemocytometer counts. The cells (approximately 10⁸) were diluted into a further 20 ml of medium and incubated overnight in a Falcon T25 tissue-culture flask to allow the attachment of adherent cells. Non-adherent cells from this culture were taken for testing with ECM molecules and various surfaces. The cells were divided among five 60 mm polystyrene dishes that had been prepared as follows: Matrigel™ (BD Biosciences, Bedford, Mass.) at dilutions of 1:4, 1:16 and 1:64 (and undiluted) was spotted onto each of the dishes using a Biomek 2000 robotic workstation equipped with a 0.45 mm diameter high density replication tool. Spots were arranged in a grid pattern. The following types dishes/surfaces were used:

1. Falcon 1007 bacteriological grade polystyrene (BD Biosciences, Bedford, Mass.)

2. Falcon 3007 tissue culture-treated polystyrene

3. Falcon 1007 dish coated with nitrocellulose dissolved in methanol and dried overnight (to provide a more protein-adhesive surface

4. Falcon 3007 dish coated with nitrocellulose (see #3)

5. Falcon 1007 dish on which HA was coated. The HA was oxidized overnight at room temperature in 50 mM sodium periodate to oxidize cis-diols to aldehyde groups.

After 2 days of culture, the plates were washed with PBS to remove nonadherent cells and the remaining adherent cells were fixed in 4% paraformaldehyde in PBS. The fixed cells were permeabilized with 1% Triton X-100, 5% neonatal goat serum in PBS for 30 min. at room temperature. Cells were then incubated in a 1:400 dilution of mouse anti-rat nestin mAb (Pharmingen Catalog #556309) for 1 hr. Cells were washed 3 times with 0.1% Triton X-100 in PBS (PBST) and further incubated with a 1:100 dilution of rhodamine-conjugated goat anti-mouse immunoglobulin antiserum (Chemicon AP181R) for 30 min. Cells were washed 3 times with PBST and mounted in Mowiol (Calbiochem). Cells were observed using a Nikon TE300 inverted microscope with epifluorescence illumination. Images were collected using a Spot camera and associated software.

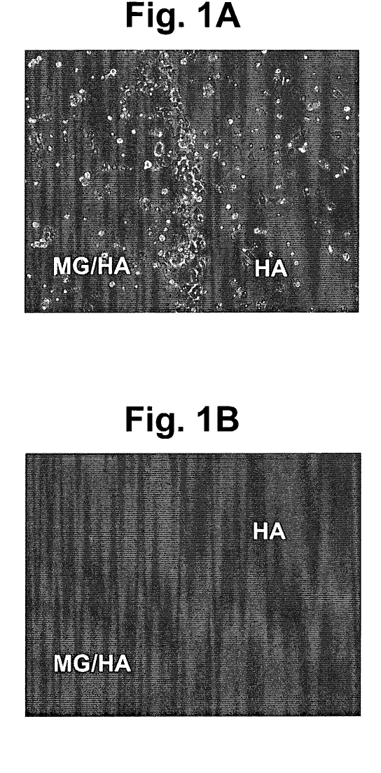

FIGS. 1A-1B depict rat BM stem cells growing on a 1:16 dilution of Matrigel as the ECM material bonded to oxidized HA. In the phase contrast micrograph shown in FIG. 1A the cells can be seen growing to a higher density on the areas of the plate displaying Matrigel than on the areas coated with HA only. FIG. 1B shows differentiation of BM cells to NSCs, as detected by visualization of nestin. Similar results (not shown) were obtained using a 1:4dilution of Matrigel

Thus, differentiation of BM stem cells into NPCs was induced by Matrigel™ at a 1:4 or 1: 16 dilution. A greater percentage of cells were differentiated at the 1:4 dilution. Increased proliferation was seen in areas of higher concentration of Matrigel™. Although some cell proliferation was observed on regions coated with HA only, it was markedly less intense than on Matrigel™-coated regions. Furthermore, there appeared to be a migration of the cells toward the Matrigel™ areas of the plate, particularly noticeable in FIG. 1A. Overall, differentiation of BM stem cells into NPCs was not detectable in the absence of ECM molecules.

TABLE 1

| Bioactive Region | | | |
|---|---|---|---|
| Factor1 | Factor2 | Differentiation | |
| (polypeptide) | (ECM molecule) | (% Nestin⁺cells)¹ | Stimulatory ?² |
| poly-L-ornithine | Laminin | 69 | Yes |
| poly-L-ornithine | Fibronectin | 52 | Yes |
| poly-L-ornithine | Collagen VI | 44 | Yes |
| poly-L-ornithine | Vitronectin | 21 | Yes |
| poly-L-ornithine | Collagen I | 13 | |
| poly-L-ornithine | Collagen III | 9 | |
| poly-L-lysine | Collagen VI | 41 | Yes |
| poly-L-lysine | Vitronectin | 31 | Yes |
| poly-L-lysine | Collagen I | 17 | |
| poly-L-lysine | Collagen IV | 11 | |
| poly-L-lysine | Fibronectin | 4 | |
| poly-L-lysine | Laminin | 10 | |
| poly-L-lysine | Collagen III | 7 | |
| poly-L-lysine | Elastin | 3 | |
| Factor1 | Factor2 | | |
| (polypeptide) | (ECM molecule) | | |
| poly-L-ornithine | poly-L-lysine | 35 | Yes |

¹% of imaged cells that stained positively for nestin
A mixture of all 10 factors-poly-L-ornithine, poly-L-lysine, fibronectin, vitronectin, collagen I, collagen III, collagen IV, collagen VI, laminin and elastin gave values in the area of 21-25%. Controls of polyamino acids alone gave results well below 20%.
²All combinations yielding ≧21% nestin positive cells are predicted to be effective stimulators of NPC differentiation.

### EXAMPLE 2

### Induction of NPC Differentiation of BM Stem Cells Treated with Combinations of A Polyamino Acid and an ECM Molecule

Studies were performed to evaluate the ability of poly-L-ornithine (PLO) and poly-L-lysine (PLL) in combination with ECM molecules to stimulate the differentiation of BM stem cells into NPC. Bioactive regions were created in 96 well microplates on a layer of HA bonded to the polystyrene surface. The agents designated as Factor 1 (PLO or PLL) and Factor 2 (either an ECM protein or, in one case, PLL) were added to wells in which the HA was oxidized by periodate as described at final concentrations of 50 μg/ml in the presence of borohydride. Unreacted material was washed away. BM stem cells (see above) were added to the wells and cultured and processed as in Example 1. The combinations tested and results are shown in Table 1.

It was concluded that the following combinations of a polypeptide (polyamino acid) and ECM molecule were effective inducers of NPC differentiation: PLO/laminin, PLO/fibronectin, PLO/collagen VI, PLO/vitronectin, PLL/collagen VI and PLL/vitronectin. Additionally, the PLO/PLL combination was effective.

### EXAMPLE 3

### Role of ECM Molecules in Promoting Growth of MC3T3 Cells

This example demonstrates use of the present method for rapid and facile ascertainment of which types and concentrations of bioactive agents effect cell proliferation and the differentiation of bone progenitor cells (osteogenic stem cells).

Murine MC3T3-E1 cells used in this example are from a clonal line of murine calvaria-derived osteoblast (Jpn. J: Oral Biol 23, 899 (1981)). The cells were cultured in α-MEM medium supplemented with 10% FBS and penicillin/streptomycin and were processed similarly to the rat bone BM cells in Example 1 through the fixation step after 11 days in culture. Cells were initially plated at a relatively low density. The presence of alkaline phosphatase enzymatic activity was detected colorimetrically using a commercial kit from Sigma Diagnostics (St. Louis, Mo.) according to the manufacturer's directions.

Proliferation of MC3T3s on oxidized HA spotted with 4 different dilutions of Matrigel™ (neat, 1:4, 1:16 and 1:64) was evaluated after five days of culture. Attachment and growth were poor at the two highest Matrigel™ concentrations. At the 1:16 and the 1:64 dilutions of Matrigel™, cells attached well and grew to a confluent monolayer over the Matrigel™ spot. Although some growth over areas of the plate lacking ECM was evident, it was markedly lower.

Growth of MC3T3 cells on different dilutions of Type I Collagen (BD Biosciences, Bedford, Mass.) was also tested. Similar to the growth results described above, less cell proliferation was evident at higher concentrations of the ECM material. There was more attachment and growth at the 1:4 dilution of Collagen Type I than that induced by the 1:4 dilution of Matrigel™. Cell attachment and growth was greater at the 1:16 and 1:64 dilutions of Collagen Type I.

### EXAMPLE 4

### Screening ECM Molecules Using Growth and Differentiation of MC3T3 Cells

This example demonstrates use of the present method for rapid and facile ascertainment of which types and concentrations of bioactive agents promote cell differentiation using MC3T3 cells. The MC3T3 cells and plates from EXAMPLE 3 were used but were incubated for an additional 6 days, for a total culture interval of 11 days. At that time, cells were fixed with formaldehyde and processed to visualize the production of alkaline phosphatase which is indicative of differentiation of MC3T3 cells to a more bone-like phenotype.

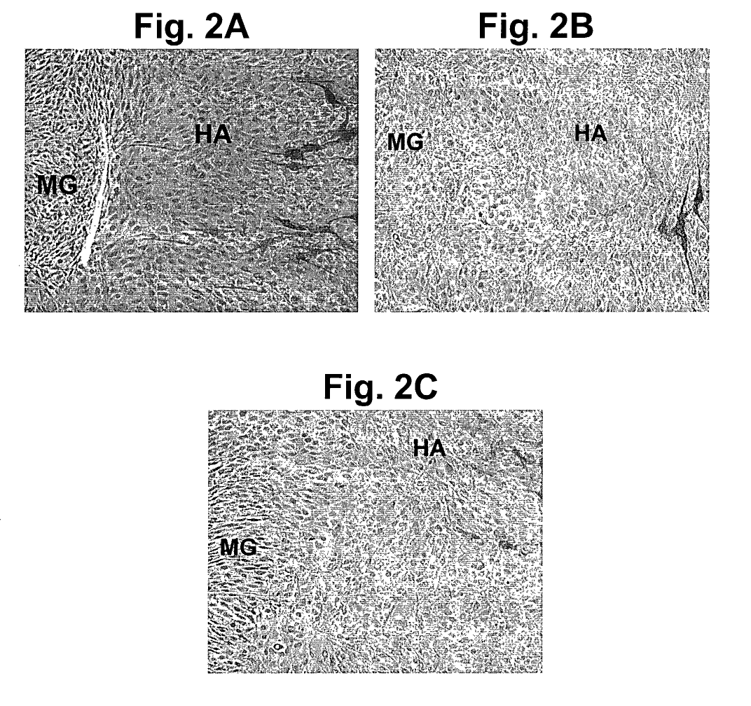

FIGS. 2A-2C show that differentiation after 11 days in culture occurred only on areas of the plate that lacked ECM molecules. Only on the surface of oxidized HA lacking ECM molecules was there differentiation into alkaline phosphatase-positive cells. Greater cell proliferation, as described in EXAMPLE 3, was only evident in the presence of ECM (here Matrigel™). Therefore, ECM molecules appear to inhibit osteogenic stem cell (here, the MC3T3 cell line) differentiation to a bone-like phenotype.

All references cited above are incorporated by reference in their entirety, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A method for evaluating a candidate agent for its cell growth-inducing or cell differentiation-inducing activity, comprising:
(a) forming a support surface coated with a cell-adhesion resistant (CAR) material and binding thereto at least one bioactive agent, resulting in a surface with at least one exposed CAR region and at least one exposed bioactive region, wherein said CAR material, said at least one bioactive agent or a combination of both are the candidate agents;
(b) adding cells to said support surface such that cells contact each of said regions; and
(c) detecting or measuring cell growth and/or differentiation on each of said regions, wherein the presence of cell growth or differentiation on a region is indicative that said candidate inducing agent present in said region has said growth-inducing or said differentiation-inducing activity.

2. The method of claim 1, wherein said at least one CAR region comprises a CAR material selected from the group consisting of (a) polyethylene glycol, (b) glyme, (c) a glyme derivative, (d) poly-HEMA, (e) polyisopropylacrylamide, (f) hyaluronic acid, (g) alginic acid and (h) a combination of any of (a)-(g).

3. The method of claim 1, wherein said at least one bioactive region comprises a bioactive agent which is an extracellular matrix molecule or a growth factor.

4. The method of claim 3, wherein said bioactive agent is an extracellular matrix molecule selected from the group consisting of laminin, vitronectin, fibronectin, elastin, collagen I, collagen III, collagen IV, collagen VI, entactin, a proteoglycan, and Matrigel™.

5. The method of claim 1 wherein said bioactive agent is an adhesion domain of an extracellular matrix molecule or other cell adhesion molecule.

6. The method of claim 1, wherein said detecting or measuring comprises visualizing the binding of an antibody to said cells.

7. The method of claim 1, wherein said at least one CAR region comprises a CAR material bonded to a tissue culture-treated surface.

8. The method of claim 1, wherein said at least one CAR material is bonded to the surface through an intermediate layer which is bonded directly to said surface.

9. The method of claim 8, wherein said intermediate layer comprises polyethyleneimine, poly-L-lysine, poly-D-lysine, poly-L-ornithine, poly-D-ornithine, poly(vinylamine), or poly(allylamine).

10. The method of claim 1 or 2, wherein said CAR material is oxidized with a mild oxidizing agent prior to adding said bioactive agent.

11. The method of claim 10 wherein said oxidizing agent is sodium periodate.

12. The method of claim 3, wherein said bioactive region comprises growth factors.

13. The method of claim 1, wherein said at least one bioactive region is in the form of a plurality of spots, with each spot comprising at least one type of bioactive molecule deposited thereon.

14. The method of claim 1, wherein said at least one bioactive region is in the form of a plurality of spots, with each spot comprising a different concentration of one type of bioactive molecule.

15. The method of claim 1, wherein said at least one bioactive region is in the form of spots in a grid pattern on said surface.

16. The method of claim 13, wherein said plurality of spots are in a grid pattern on said surface.

17. The method of claim 14, wherein said plurality of spots are in a grid pattern on said surface.

18. The method of claim 13 wherein said bioactive molecule is an extracellular matrix molecule.

19. The method of claim 14 wherein said bioactive molecule is an extracellular matrix molecule.

20. The method of claim 1, wherein said at least one bioactive region is in the form of a plurality of wells, with each well comprising at least one type of bioactive molecule deposited thereon.

21. The method of claim 20 wherein said bioactive molecule is an extracellular matrix molecule.

22. The method of claim 1, wherein said cells are stem cells.

23. The method of claim 22, wherein said stem cells are pluripotent stem cells.

24. The method of claim 22, wherein said stem cells are progenitor stem cells.

25. The method of claim 22, wherein said stem cells are embryonic stem cells.

26. The method of claim 22, wherein said stem cells are hematopoietic stem cells.

27. The method of claim 22, wherein said stem cells are bone marrow-derived stem cells.

28. The method of claim 22, wherein said stem cells are osteogenic stem cells.

29. The method of claim 22 wherein said stem cells differentiate into neuronal progenitor cells.

30. A method for inducing or promoting the differentiation of stem cells into neuronal progenitor cells comprising:
(a) contacting stem cells in a culture vessel having a surface coated with one or more cell adhesion resisting (CAR) agents and one or more bioactive agents that induce stem cell differentiation to neural progenitor cells; and
(b) culturing said stem cells for a time sufficient to permit them to differentiate into neuronal progenitor cells.

31. The method of claim 30 further comprising
(c) detecting said differentiation.

32. The method of claim 30 wherein said bioactive agents are extracellular matrix molecules.

33. The method of claim 30 wherein said bioactive agents are a combination of a polycationic polyamino acid and an extracellular matrix molecule.

34. The method of claim 33 wherein said combination is selected from the group consisting of: (a) poly-omithine and laminin; (b) poly-omithine and fibronectin; (c) poly-omithine and collagen VI; (d) poly-omithine and vitronectin; (e) poly-lysine and collagen VI; (f) poly-lysine and vitronectin; and (g) poly-ornithine and poly-lysine.

35. A method for inducing or promoting the differentiation of stem cells into neuronal progenitor cells comprising:
(a) contacting stem cells in a culture vessel having a surface with a combination of juxtaposed regions of different bioactive agents or different concentrations of a bioactive agent, which combination is capable of inducing stem cell differentiation to neural progenitor cells; and
(b) culturing said stem cells for a time sufficient to permit them to differentiate into neuronal progenitor cells.

36. The method of claim 35 further comprising
(c) detecting said differentiation.

37. The method of claim 35 wherein said bioactive agents are extracellular matrix molecules.

38. A method for producing an isolated or enriched population of neuronal progenitor cells from stem cells, comprising:
(a) inducing or promoting the differentiation of stem cells into neuronal progenitor cells in accordance with claim 30, thereby producing said neuronal progenitor cells;
(b) optionally, detecting said differentiation; and
(c) enriching or isolating said neuronal progenitor cells.

39. The method of claim 28, wherein said bioactive agents are extracellular matrix molecules.

40. A method for producing an isolated or enriched population of neuronal progenitor cells, comprising:
(a) inducing or promoting the differentiation of stem cells into neuronal progenitor cells in accordance with claim 35, thereby producing said neuronal progenitor cells;
(b) optionally, detecting said differentiation; and
(c) enriching or isolating said neuronal progenitor cells.

41. The method of claim 40, wherein said bioactive agents are extracellular matrix molecules.

42. The method of any of claims 30, 35, 38 or 40 wherein the CAR material is selected from the group consisting of (a) polyethylene glycol, (b) glyme, (c) a glyme derivative, (d) poly-HEMA, (e) polyisopropylacrylamide, (f) hyaluronic acid, (g) alginic acid and (h) a combination of any of (a)-(g).

43. The method of claim 42 wherein said CAR material is hyaluronic acid.

44. The method of any of claims 30, 35, 38 or 40, wherein said CAR material is bonded to an intermediate layer which is bonded to said surface.

45. The method of claim 44 wherein said intermediate layer is selected from the group consisting of polyethyleneimine, poly-L-lysine, poly-D-lysine, poly(vinylamine), and poly(allylamine).

46. The method any of claims 30, 35, 38 or 40 wherein said cells are stem cells.

47. The method claim 46, wherein said stem cells are bone marrow stem cells.

48. The method claim 46, wherein said stem cells are pluripotent stem cells.

49. The method of claim 31 or 36, wherein said detecting step comprises detecting or measuring nestin in or on cells that have differentiated to neuronal progenitor cells.

50. The method of claim 42 wherein nestin is detected or measured using an antibody specific for nestin.

51. An article useful for evaluating a candidate agent for its cell growth-inducing or cell differentiation-inducing activity, comprising:
(a) a support surface coated with a cell-adhesion resistant (CAR) material having bound thereto at least one bioactive agent, such that the resulting surface comprises at least one exposed CAR region and at least one exposed bioactive region, wherein said CAR material, said at least one bioactive agent or a combination of both is the candidate agent.

52. The article of claim 51, wherein said at least one CAR region comprises a CAR material selected from the group consisting of (a) polyethylene glycol, (b) glyme, (c) a glyme derivative, (d) poly-HEMA, (e) polyisopropylacrylamide, (f) hyaluronic acid, (g) alginic acid and (h) a combination of any of (a)-(g).

53. The article of claim 51, wherein said at least one CAR region comprises a tissue culture-treated surface to which said CAR material is bonded.

54. The article of claim 51, wherein said at least one bioactive region comprises a bioactive agent which is an extracellular matrix molecule or a growth factor.

55. The article of claim 53, wherein said at least one bioactive region comprises extracellular matrix molecules selected from the group consisting of laminin, vitronectin, fibronectin, elastin, collagen I, collagen III, collagen IV, collagen VI, entactin, a proteoglycan, or Matrigel™.

56. The article of claim 51, wherein said at least one CAR region is bonded to the surface through an intermediate layer which is bonded directly to said surface.

57. The article of claim 56, wherein said intermediate layer comprises polyethyleneimine, poly-L-lysine, poly-D-lysine, poly(vinylamine), or poly(allylamine).

58. The article of claim 54, wherein said bioactive region comprises growth factors selected from the group consisting of a bone morphogenetic proteins, epidermal growth factor, erythropoietin, heparin binding factor, hepatocyte growth factor, insulin, insulin-like growth factor I or II, an interleukin, a muscle morphogenic protein, nerve growth factor, platelet-derived growth factor, and transforming growth factor α or β.

59. The article of claim 51, wherein said at least one bioactive region is in the form of a plurality of spots, with each spot comprising at least one bioactive agent deposited thereon.

60. The article of claim 51, wherein said at least one bioactive region is in the form of a plurality of spots, with each spot comprising a different concentration of one bioactive agent.

61. The article of claim 59 or 60, wherein said plurality of spots are arrayed in a grid pattern on said surface.
